(19) United States
(12) Patent Application Publication
(10) Pub. No.: US 2024/0065951 A1
(43) Pub. Date: Feb. 29, 2024

(54) SKIN-LIFTING COSMETIC COMPOSITION FOR ANTI-AGING AND PHOTOTHERMAL SKIN CARE METHOD USING SAME

(71) Applicant: SIMFLE STICK INC., Daegu (KR)

(72) Inventor: Chang Hoon KIM, Daegu (KR)

(73) Assignee: SIMFLE STICK INC., Daegu (KR)

(21) Appl. No.: 18/260,057

(22) PCT Filed: Jul. 14, 2021
(86) PCT No.: PCT/KR2021/009042

§ 371 Date: Jun. 30, 2023

(30) Foreign Application Priority Data
Jan. 7, 2021 (KR) 10-2021-0001844

(51) Int. Cl. A61K 8/19; A61K 8/02; A61K 8/25; A61K 8/36; A61K 8/86; A61Q 19/08

(52) U.S. Cl. CPC A61K 8/19; A61K 8/0245; A61K 8/25; A61K 8/36; A61K 8/86; A61Q 19/08; A61K 2800/24; A61K 2800/47; A61K 2800/621; A61K 2800/624; A61K 2800/651; A61K 2800/81; A61K 2800/82; A61K 2800/884

(57) ABSTRACT

The present invention relates to a skin-lifting cosmetic composition for anti-aging and a photothermal skin care method using same, the cosmetic composition containing, as an active ingredient, core-shell particles comprising: a magnetic core; a first shell layer surrounding the magnetic core and containing a dielectric substance; a second shell layer surrounding the first shell layer and containing a metal; and a third shell layer surrounding the second shell layer and containing a hydrophilic polymer. The skin-lifting cosmetic composition for anti-aging according to the present invention is applied to a photothermal skin care method comprising a step in which the core-shell structured microparticles, including a gold (Au) nanoparticle layer and contained as an active ingredient, infiltrate into follicles, sebaceous glands, pilosebaceous ducts, and infundibula of facial skin through ultrasound application and then are heated through light irradiation, whereby the cosmetic composition exhibits an enhanced skin lifting effect as well as outstanding skin anti-aging effects encompassing skin elasticity improvement, skin laxity reduction, and skin wrinkle relief.

### TECHNICAL FIELD

The present disclosure relates to an anti-aging cosmetic composition and a skin care method using the same. More particularly, the present disclosure relates to a skin-lifting cosmetic composition and a photothermal skin care method using the same.

### BACKGROUND ART

As economic development continues, individuals have higher economic levels and greater interest in pursuing happiness and anti-aging. As a result, research is underway to delay both intrinsic and extrinsic aging of the body using various methods.

In terms of extrinsic aging, the field of skin is given the most attention. In particular, the facial skin is a part constituting the largest portion where others can see, playing an important role in expressing one's beauty. Cosmetics, used as a means for expressing beauty, have aesthetic functions as well as functions of inhibiting causes of skin aging, such as free radicals attributable to extrinsic factors, such as environmental pollutants, stress, ultraviolet rays, etc., and intrinsic factors, such as immune cell malfunction, cell activity degradation, etc., active oxygen, proteins regulated by peroxides and the like, nucleic acids, degradation of cellular membrane lipids, and the like, are regarded as important.

On the other hand, skin aging is divided into intrinsic and extrinsic aging based on biological processes. Intrinsic aging refers to chronological aging that occurs over time. Extrinsic aging includes photoaging occurring when all kinds of pollutants and ultraviolet rays lead to skin thinning and a decrease in elasticity, and when keratinocytes and melanocytes in the epidermis have cell damage by ultraviolet B (UVB) as exposed to ultraviolet irradiation.

As the skin ages, the amounts and arrangements of collagen, elastin, hyaluronic acid, and glycoprotein, which are substances constituting the skin, change or decrease. In addition, free radicals and harmful active oxygen give oxidative stress. Furthermore, due to aging or ultraviolet rays, biosynthesis of cyclooxygenase-2 (Cox-2), an enzyme that produces proinflammatory cytokines known to cause inflammation, increases in most cells constituting the skin. Moreover, biosynthesis of matrix metalloprotease (MHP), an enzyme that decomposes skin tissue using inflammatory factors thereof, increases, and nitric oxide (NO) production by inducible nitric oxide synthase (iNOS) is known to increase.

That is, there are decreases in cell activity according to naturally occurring endogenous aging as well as decreases in the biosynthesis of matrix substances due to microinflammation. In addition, there are external factors, such as stress increased due to a variety of adverse circumstances and active oxygen species increased due to sunlight. As a result, degradation and denaturation are accelerated, so the skin matrix is removed while the thickness thereof shrinks, and symptoms of skin aging thus develop.

Accordingly, research is in progress on anti-aging active ingredients and anti-aging skin care methods capable of preventing and relieving signs of skin aging as described above.

### DISCLOSURE

### Technical Problem

An objective of the present disclosure is to provide a novel skin-lifting cosmetic composition for anti-aging and a photothermal skin care method that is specifically designed for the cosmetic composition and capable of more immediately exhibiting a skin-lifting effect than conventional technology.

### Technical Solution

In order to accomplish the above objective, the present disclosure provides a skin-lifting cosmetic composition for anti-aging, the composition containing core-shell particles as an active ingredient. The core-shell particles include: a magnetic core; a first shell layer surrounding the magnetic core and including a dielectric substance; a second shell layer surrounding the first shell layer and including one or more metals selected from the group consisting of gold (Au), silver (Ag), and platinum (Pt); and a third shell layer surrounding the second shell layer and including a hydrophilic polymer.

In this case, the skin-lifting cosmetic composition for anti-aging may include the core-shell particles in which the dielectric substance included in the first shell layer is silica, the metal included in the second shell layer is gold (Au), and the hydrophilic polymer included in the third shell layer is polyethylene glycol (PEG).

In addition, the skin-lifting cosmetic composition for anti-aging may contain the core-shell particles in an amount of 0.1 mg/mL to 10 mg/mL based on the total volume of the cosmetic composition.

Furthermore, another aspect of the present disclosure provides a skin care method using the skin-lifting cosmetic composition for anti-aging. The method includes: (a) peeling facial skin; (b) applying the skin-lifting cosmetic composition for anti-aging on the surface of the facial skin; (c) primarily facilitating infiltration of core-shell particles contained in the cosmetic composition into follicles, sebaceous glands, pilosebaceous ducts, and infundibula of the skin by applying ultrasound to the facial skin coated with the cosmetic composition; (d) secondarily facilitating infiltration of the core-shell particles contained in the cosmetic composition into the follicles, sebaceous glands, pilosebaceous ducts, and infundibula of the skin by attaching a polymer film mask to the facial skin coated with the cosmetic composition; (e) removing residues of the cosmetic composition from the surface of the facial skin with a laser beam; and (f) heating the core-shell particles infiltrated into the follicles, sebaceous glands, pilosebaceous ducts, and infundibula of the skin by irradiating the cosmetic composition-removed facial skin with light.

In this case, when performing the facial skin peeling in the (a), superficial chemical peeling using trichloroacetic acid (TCA) may be used.

In addition, when removing the residues of the cosmetic composition containing the core-shell particles that fail to be infiltrate pores and the like from the surface of the skin in the (e), a method of irradiating the facial skin with a Q-switched Nd:YAG laser beam may be used.

### Advantageous Effects

A skin-lifting cosmetic composition for anti-aging, according to the present disclosure, is applied to a photothermal skin care method including steps in which core-shell structured microparticles, contained as an active ingredient and including a nanoparticle layer of any one or more metals selected from the group consisting of gold (Au), silver (Ag), and platinum (Pt), are infiltrated into follicles, sebaceous glands, pilosebaceous ducts, and infundibula of the facial skin by ultrasound application and the like, and then are heated by light irradiation. Thus, the cosmetic composition exhibits an enhanced skin-lifting effect as well as outstanding anti-aging effects encompassing skin elasticity improvement, skin laxity reduction, and skin wrinkle reduction.

### DESCRIPTION OF DRAWINGS

FIG. 1 shows a flowchart sequentially illustrating each step included in a photothermal skin care method using a skin-lifting cosmetic composition for anti-aging according to the present disclosure;

FIG. 2 shows the conditions of a test subject before conducting an experiment (left) and after performing the treatment two times using a method of the present disclosure (right);

FIG. 3 shows the skin conditions of each patient before performing a treatment and after performing the treatment one time and two times;

FIG. 4 shows an image of a tissue test on the back of a biopsy subject where gold is delivered into pores and shrinks the pores by converting a laser beam into heat;

FIGS. 5 and 6 show hematoxylin-eosin (H&E) staining images of the pores before and after performing the treatment, respectively;

FIG. 7 shows tissue changes to confirm the effect of the occlusive dressing technique (ODT) in the present disclosure, the changes observed when irradiating a hairless mouse with only ultrasound (left) and a laser beam in a combination of laser application and ODT treatment (right); and

FIGS. 8 and 9 show transmission electron microscopy (TEM) images of particles coated with only silica and particles coated with gold on top of a silica layer, respectively.

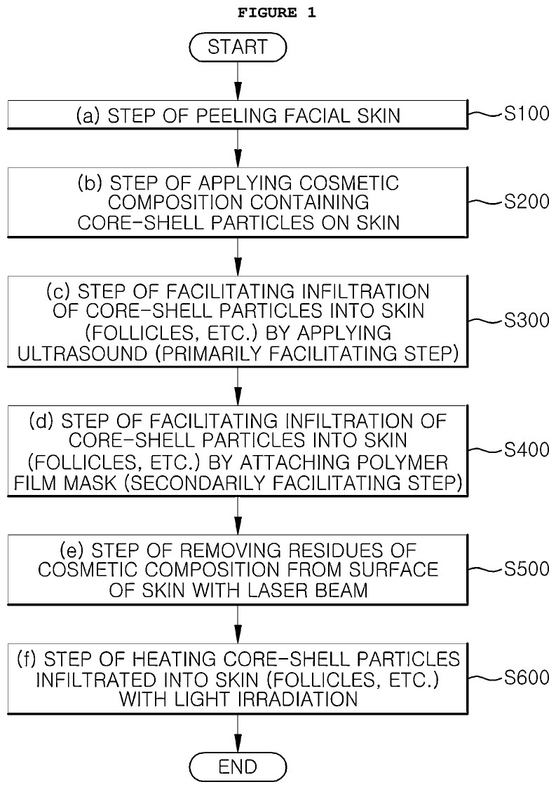

### BEST MODE

In the following description of the present disclosure, detailed descriptions of known functions and components incorporated herein will be omitted when it may make the subject matter of the present disclosure unclear.

Reference will now be made in detail to various embodiments of the present disclosure, specific examples of which are illustrated in the accompanying drawings and described below, since the embodiments of the present disclosure can be variously modified in many different forms. While the present disclosure will be described in conjunction with exemplary embodiments thereof, it is to be understood that the present description is not intended to limit the present disclosure to those exemplary embodiments. On the contrary, the present disclosure is intended to cover not only the exemplary embodiments, but also various alternatives, modifications, equivalents, and other embodiments that may be included within the spirit and scope of the present disclosure as defined by the appended claims.

The terminology used herein is for the purpose of describing particular embodiments only and is not intended to be limiting. As used herein, the singular forms are intended to include the plural forms as well, unless the context clearly indicates otherwise. It will be further understood that the terms “comprise”, “include”, “have”, etc. when used herein, specify the presence of stated features, integers, steps, operations, elements, components, and/or combinations of them but do not preclude the presence or addition of one or more other features, integers, steps, operations, elements, components, and/or combinations thereof.

Hereinafter, the present disclosure will be described in detail.

In the present disclosure, a skin-lifting cosmetic composition for anti-aging contains core-shell particles having a particular laminated structure as an active ingredient. The core-shell particles are characterized by including: a magnetic core, a first shell layer surrounding the magnetic core and including a dielectric substance, a second shell layer surrounding the first shell layer and including one or more metals selected from the group consisting of gold (Au), silver (Ag), and platinum (Pt), and a third shell layer surrounding the second shell layer and including a hydrophilic polymer.

The magnetic core is magnetic nanoparticles or a magnetic nanocluster, which is a collection of the magnetic nanoparticles, present in a dispersed form in particles or in the form of a core. In addition, the magnetic substance has a diameter in a range of 1 nm to 1000 nm.

The first shell layer including the dielectric substance may have a thickness in a range of 5 nm to 50 nm. In addition, the dielectric substance included in the first shell layer may be selected from the group consisting of silica, titania, polymer particles, alumina, and mixtures thereof.

The second shell layer including the metal includes a metal selected from the group consisting of Au, Ag, and Pt, and may have a thickness in a range of 10 nm to 50 nm.

The third shell layer including the hydrophilic polymer may include polyethylene glycol (PEG) or polypropylene glycol (PPG) as the hydrophilic polymer.

On the other hand, the core-shell particles include: the magnetic core; the first shell layer surrounding the magnetic core and including the dielectric substance; and the second shell layer surrounding the first shell layer and including the one or more metals selected from the group consisting of gold (Au), silver (Ag), and platinum (Pt). As a result, electromagnetic waves may be absorbed due to the surface plasmon resonance phenomenon, and the absorbed electromagnetic waves generate heat by collectively vibrating conductive electrons in a metal layer. A wavelength range of the electromagnetic waves being absorbed due to the core-shell particles and the generated heat thereby may be easily controlled by adjusting the types of metal being used, the thickness of the shell layer including the dielectric substance, the thickness of the magnetic core and the like. In addition, electromagnetic waves are characterized by being capable of penetrating biological tissue to some extent, although the degree of penetration varies depending on the wavelength.

The core-shell particles may be contained in an amount in a range of 0.1 mg/mL to 10 mg/mL based on the total volume of the cosmetic composition, and further preferably in the range of 0.3 mg/mL to 5.0 mg/mL based on the total volume of the cosmetic composition. When the amount of the core-shell particles is smaller than 0.1 mg/mL, the efficacy thereof is marginal. When the amount of the core-shell particles exceeds 10 mg/mL, the stability and safety in formulation are problematic, and the usage itself is cost-ineffective.

On the other hand, a method of preparing the core-shell particles is not particularly limited. The core-shell particles may, for example, be prepared according to a method disclosed in Korean Patent No. 10-0924786.

The skin-lifting cosmetic composition for anti-aging, according to the present disclosure, may be prepared in one formulation selected from the group consisting of a toner, a lotion, a serum, an essence, a facial mask, a UV cream, BB cream, a nourishing cream, a massage cream, a cleansing cream, a facial wash, a cleansing water, a powder, and a hand wash.

That is, the cosmetic composition of the present disclosure may be prepared in any formulation commonly prepared in the art. For example, the cosmetic composition may be formulated as a solution, a suspension, an emulsion, a paste, a gel, a cream, a lotion, a powder, a soap, a surfactant-containing cleanser, an oil, a powder foundation, an emulsion foundation, a wax foundation, and spray, but is not limited thereto. That is, the cosmetic composition may be further specifically made into a toner, a lotion, a serum, an essence, a facial mask, a UV cream, a BB cream, a nourishing cream, a massage cream, a cleansing cream, a facial wash, a cleansing water, powder, and hand wash.

In addition, the cosmetic composition may be prepared in the form of an adjuvant which is commonly used in the field of dermatology and is locally or generally applicable by separately incorporating a dermatologically acceptable medium or base, other than cosmetics containing the active ingredient of the core-shell particles of the present disclosure. Cosmetics may, for example, be provided in the form of a proper formulation of a solution, a gel, a solid or pasty anhydrous product, an emulsion obtained by dispersing an oil phase in an aqueous phase, a suspension, a microemulsion, microcapsules, microgranules, ionic type (liposome) or a non-ionic vascular dispersant, a cream, a toner, a lotion, a powder, an ointment, a spray, or a concealing stick.

In addition, the cosmetic composition of the present invention may include a fatty material, an organic solvent, a dissolving agent, concentrating and gelling agents, a softening agent, an antioxidant, a suspending agent, a stabilizer, a foaming agent, a flavoring agent, a surfactant, water, an ionic or nonionic emulsifier, a filler, sequestering and chelating agents, a preservative, a vitamin, a blocking agent, a wetting agent, an essential oil, a dye, a piment, a hydrophilic or lipophilic activating agent, a lipid vesicle, or an adjuvant commonly used in the field of cosmetology or dermatology, such as other components commonly used for cosmetics, in addition to the active ingredient of the core-shell particles.

Furthermore, the components described above may be introduced in an amount generally used in the dermatology field. Examples of products to which the cosmetic composition of the present disclosure is applicable may include cosmetics such as an astringent toner, a softening toner, a nourishing toner, all types of creams, an essence, a face mask, a foundation, and the like, a cleanser, a face wash, a soap, a treatment, a cosmetic solution, and the like. Specific formulations of the cosmetic composition of the present disclosure include a skin lotion, a skin softer, a skin toner, an astringent, a lotion, a milk lotion, a moisturizing lotion, a nourishing lotion, a massage cream, a nourishing cream, a moisturizing cream, a hand cream, an essence, a nourishing essence, a face mask, a soap, a shampoo, a face wash, a cleansing lotion, a cleansing cream, a body lotion, a body cleanser, an emulsion, a pressed powder, a loose powder, an eye shadow, and the like.

Specifically, when the formulation of the present disclosure is the paste, the cream, or the gel, animal oil, vegetable oil, wax, paraffin, starch, tragacanth, a cellulose derivative, polyethylene glycol, silicone, bentonite, silica, talc, zinc oxide, or the like may be used as a carrier component.

When the formulation of the present disclosure is the solution or the emulsion, a solvent, a dissolving agent, or an emulsifier is used as a carrier component, which, for example, includes water, ethanol, isopropanol, ethyl carbonate, ethyl acetate, benzyl alcohol, benzyl benzoate, propylene glycol, 1,3-butyl glycol oil, glycerol aliphatic ester, polyethylene glycol, or fatty acid ester of sorbitan.

When the formulation of the present disclosure is the suspension, a liquid diluent, such as water, ethanol, or propylene glycol, a suspending agent, such as ethoxylated isostearyl alcohol, polyoxyethylene sorbitol ester, and polyoxyethylene sorbitan ester, microcrystalline cellulose, aluminum metahydroxide, bentonite, agar, tragacanth, or the like may be used as a carrier component.

When the formulation of the present disclosure is the powder or the spray, lactose, talc, silica, aluminum hydroxide, calcium silicate, or polyamide powder may be used as a carrier component. In particular, in the case of the spray, a propellant such as chlorofluorohydrocarbon, propane/butane, or dimethyl ether may be additionally included. When the formulation of the present disclosure is the surfactant-containing cleanser, aliphatic alcohol sulfate, aliphatic alcohol ether sulfate, sulfosuccinic acid monoester, isethionate, an imidazolinium derivative, methyl taurate, sarcosinate, fatty acid amide ether sulfate, alkylamidobetaine, aliphatic alcohol, fatty acid glyceride, fatty acid diethanolamide, vegetable oil, a lanolin derivative, ethoxylated glycerol fatty acid ester, or the like may be used as a carrier component.

When the cosmetic composition of the present disclosure has the formulation of the soap, the surfactant-containing cleanser, or the surfactant-free cleanser, the cosmetic composition may be applied on the skin and then wiped off, removed, or washed with water. As specific examples, the soap is a liquid soap, a powder soap, a solid soap, and an oil soap, the surfactant-containing cleanser is a face wash, a cleansing water, a cleansing towel, and a cleansing mask, and the surfactant-free cleanser is a cleansing cream, a cleansing lotion, a cleansing water, and a cleansing gel, but are not limited thereto.

On the other hand, a cosmetic method of the present disclosure refers to all cosmetic methods using the cosmetic composition of the present disclosure. In other words, the cosmetic method of the present disclosure includes all known methods in the art that use cosmetic compositions. The cosmetic composition of the present disclosure may be used alone or together, or used in combination with cosmetic compositions other than the cosmetic composition of the present disclosure. In addition, the cosmetic composition may be used according to general instructions, and the frequency of use may vary depending on skin condition or user preference.

Another aspect of the present disclosure provides a photothermal skin care method for skin lifting using the skin-lifting cosmetic composition for anti-aging, in which the cosmetic composition contains the core-shell particles as the active ingredient.

The photothermal skin care method for skin lifting according to the present disclosure is characterized by including: (a) peeling facial skin (S100); (b) applying the cosmetic composition described above on the surface of the facial skin (S200); (c) primarily facilitating infiltration of core-shell particles contained in the cosmetic composition into follicles, sebaceous glands, pilosebaceous ducts, and infundibula of the skin by applying ultrasound to the facial skin coated with the cosmetic composition (S300); (d) secondarily facilitating infiltration of the core-shell particles contained in the cosmetic composition into the follicles, sebaceous glands, pilosebaceous ducts, and infundibula of the skin by attaching a polymer film mask to the facial skin coated with the cosmetic composition (S400); (e) removing residues of the cosmetic composition from the surface of the facial skin with a laser beam (S500); and (f) heating the core-shell particles infiltrated into the follicles, sebaceous glands, pilosebaceous ducts, and infundibula of the skin by irradiating the cosmetic composition-removed facial skin with light (S600).

The (a) is a step of peeling the facial skin so that the core-shell particles, the active ingredient of the skin-lifting cosmetic composition for anti-aging according to the present disclosure, can be well delivered to the follicles, sebaceous glands, pilosebaceous ducts, and infundibula of the skin of a subject. For example, a 10% aqueous solution of trichloroacetic acid (TCA) may be used through superficial chemical peeling to peel the facial skin of a subject so that the core-shell particles, the active ingredient of the cosmetic composition, can be well delivered to follicles and the like.

In the (b), all types of formulations (a toner, a lotion, a serum, an essence, a face mask, a UV cream, a BB cream, a nourishing cream, a massage cream, a cleansing cream, a face wash, a cleansing water, a powder, and the like) containing the core-shell particles, the active ingredient of the skin-lifting cosmetic composition for anti-aging according to the present disclosure, are applied on the surface of the facial skin. In this case, in addition to the cosmetics containing the core-shell particles, ethanol, propylene glycol, a surfactant, acetone, and the like may be included in the application step to facilitate the infiltration into the skin.

Next, the (c) is a step of primarily facilitating the infiltration of the core-shell particles, the active ingredient, into the skin. The ultrasound is applied to the facial skin coated with the cosmetic composition to facilitate the infiltration of the core-shell particles contained in the cosmetic composition into the follicles, sebaceous glands, pilosebaceous ducts, and infundibula of the skin.

More specifically, in the (c), using the ultrasound with a frequency in a range of 20 kHz to 200 kHz, the facial skin coated with the cosmetic composition containing the core-shell particles may be rubbed with an output in a range of 0.1 W/cm2 to 5 W/cm2 for 5 minutes to 10 minutes.

For reference, a follicle in human skin is estimated to have a typical pore size in a range of 12 microns to 300 microns. Accordingly, an ultrasonic frequency is preferably in a range of 20 kHz to 200 kHz, as described above, and more preferably in the range of 20 kHz to 100 kHz, 20 kHz to 60 kHz, or 30 kHz to 50 kHz.

On the other hand, in the case of using ultrasound, high skin temperature is often involved, but heating to a temperature of up to about 42° C. may help deliver the cosmetic composition containing the core-shell particles to follicles. However, excessive heating is undesirable and may cause pain, tissue damage, and burns, so a coupling gel and the like may be used locally. For example, ultrasound in a low-frequency range may be used to prevent excessive heating. The skin that receives ultrasonic energy is occasionally required to be cooled by pausing the ultrasound.

The (d) is a step of secondarily facilitating the infiltration of the core-shell particles, the active ingredient for further facilitation. The polymer film mask made of polymers, such as polyethylene (PE), polypropylene (PP), and the like, is attached to the facial skin coated with the cosmetic composition for a predetermined time to facilitate the infiltration of the core-shell particles contained in the cosmetic composition into the follicles, sebaceous glands, pilosebaceous ducts, and infundibula of the skin.

Subsequently, in the (e), the cosmetic composition containing the residues of the core-shell particles is removed from the surface of the facial skin of the subject, having undergone the primarily and secondarily facilitating steps, with the laser beam used as a removal means.

For example, the facial skin of the subject is irradiated with a Q-switched Nd:YAG laser beam so that the cosmetic composition containing the residues of the core-shell particles that fail to be infiltrated into the follicles and the like is scattered and then removed from the facial skin of the subject.

Lastly, in the (f), the cosmetic composition-removed facial skin is irradiated with the light so that the core-shell particles infiltrated into the follicles, sebaceous glands, pilosebaceous ducts, and infundibula of the skin are heated to exhibit a skin-lifting effect.

For example, gold nanoparticles are considered nanomaterials and are thus positioned between dead skin cells. In the case of performing treatment using a laser, high-temperature heat of 60 degrees Celsius or more is generated. When the gold is incompletely removed, burns may occur on the surface of the skin, so the gold needs to be present only in the pores, and the surface gold needs to be removed before irradiating the surface of the skin outside pores with a laser beam. Even though being wiped with gauze in the related art, there was a problem of incompletely removing the gold. The present disclosure is characterized by using the Q-switched Nd:YAG laser beam as a method of removing the surface gold.

In this step, either a diode laser, an IPL laser, or light energy in a specific wavelength range is used as an irradiation light. In the case of the diode laser, the heating is performed by being irradiated with an energy in a range of 20 J/cm² to 33 J/cm² one to two times under conditions: a pulse duration of 30 milliseconds and an overlap of 10%. In the case of the IPL laser, the heating is performed by being irradiated with an energy in a range of 20 J/cm² to 35 J/cm² one to two times using a tip with a spot size in a range of 5 mm to 15 mm with a pulse duration of 30 ms. In the case of the light energy, the heating is performed by being irradiated with a light wavelength in a range of 400 nm to 1100 nm one to two times.

Either the diode laser, the IPL laser, or the light energy in a specific wavelength range is selected, as described above, to heat the core-shell particles delivered to the follicles, sebaceous glands, pilosebaceous ducts, and infundibula of the skin. As a result, cell temperatures are locally raised, thereby inducing the skin-lifting effect.

In addition, to obtain a further desirable skin lifting effect, the steps constituting the photothermal skin care method for skin lifting may be completely or partially performed multiple times within a predetermined period in consideration of skin conditions.

For example, after performing the photothermal skin care method once for the first time, the steps, from the peeling of the skin to the heating of the core-shell particles, may be performed one more time when one week has elapsed. Then, only the heating of the core-shell particles may be performed for three weeks to perform additional treatment with a 4-week cycle. More preferably, the additional treatment with the 4-week cycle may be performed one more time so that the additional treatment in eight weeks can maximize the skin-lifting effect.

### MODE FOR INVENTION

Hereinafter, the present specification will be described in more detail with reference to embodiments.

However, the embodiments according to the present specification may be modified in many different forms, and the scope of the present specification is not construed as being limited to the embodiments described below. The embodiments of the present disclosure described hereinbelow are provided for allowing those skilled in the art to more clearly comprehend the present disclosure.

### <Example 1> Preparation of Cosmetic Composition Containing Core-Shell Microparticles Including Gold (Au) Nanoparticle Layer

(1) Formulation Examples of toners among skin-lifting cosmetic compositions for anti-aging, that is, cosmetics containing core-shell particles composed of a magnetic core (magnetite (Fe₃O₄) layer), a first shell layer (silica layer), a second shell layer (gold (Au) nanoparticle layer), and a third shell layer (PEG layer) as an active ingredient were prepared as shown in Table 1 below.

TABLE 1

| Components | | |
|---|---|---|
| Formulation | Comparative Formulation | |
| Example 1 | Example 1 | Content (wt %) |
| Core-shell particles | — | 3.0 |
| Glycerine | Glycerine | 5.0 |
| 1,3-Butylene glycol | 1,3-Butylene glycol | 3.0 |
| PEG 1500 | PEG 1500 | 1.0 |
| Allatoin | Allatoin | 0.1 |
| DL-Panthenol | DL-Panthenol | 0.3 |
| EDTA-2Na | EDTA-2Na | 0.02 |
| Benzopyrene-9 | Benzopyrene-9 | 0.04 |
| Sodium hyaluronate | Sodium hyaluronate | 5.0 |
| Ethanol | Ethanol | 10.0 |
| Octyldodeceth-16 | Octyldodeceth-16 | 0.2 |
| Polysorbate 20 | Polysorbate 20 | 0.1 |
| Preservative, | Preservative, | Small amount |
| fragrance, pigment | fragrance, pigment | |
| Distilled water | Distilled water | Residual amount |

| | |
|---|---|
| Total | 100 |

(2) Formulation Examples of creams among skin-lifting cosmetic compositions for anti-aging, that is, cosmetics containing core-shell particles composed of a magnetic core (magnetite (Fe₃O₄) layer), a first shell layer (silica layer), a second shell layer (gold (Au) nanoparticle layer), and a third shell layer (PEG layer) as an active ingredient were prepared as shown in Table 2 below.

TABLE 2

| Components | | |
|---|---|---|
| Formulation | Comparative Formulation | |
| Example 2 | Example 2 | Content (wt %) |
| Core-shell particles | — | 3.0 |
| Lipophilic glycerine | Lipophilic glycerine | 2.0 |
| monostearate | monostearate | |
| Cetearyl alcohol | Cetearyl alcohol | 2.0 |
| Stearic acid | Stearic acid | 1.3 |
| Allatoin | Allatoin | 1.0 |
| Beeswax | Beeswax | 1.5 |
| Polysorbate 60 | Polysorbate 60 | 0.5 |
| Hydrogenated vegetable | Hydrogenated vegetable | 1.0 |
| oil | oil | |
| Squalene | Squalene | 2.5 |
| Minerals | Minerals | 5.0 |
| Trioctanoin | Trioctanoin | 4.5 |
| Dimethicone | Dimethicone | 1.5 |
| Sodium magnesium | Sodium magnesium | 0.1 |
| silicate | silicate | |
| Glycerine | Glycerine | 5.0 |
| Betaine | Betaine | 3.0 |
| Trimethanolamine | Trimethanolamine | 1.0 |
| Sodium hyaluronate | Sodium hyaluronate | 5.0 |
| Preservative, | Preservative, | Small amount |
| fragrance, pigment | fragrance, pigment | |
| Distilled water | Distilled water | Residual amount |

| | |
|---|---|
| Total | 100 |

(3) Formulation Examples of essences among skin-lifting cosmetic compositions for anti-aging, that is, cosmetics containing core-shell particles composed of a magnetic core (magnetite (Fe₃O₄) layer), a first shell layer (silica layer), a second shell layer (gold (Au) nanoparticle layer), and a third shell layer (PEG layer) as an active ingredient were prepared as shown in Table 3 below.

TABLE 3

| Components | | |
|---|---|---|
| Formulation | Comparative Formulation | |
| Example 3 | Example 3 | Content (wt %) |
| Core-shell particles | — | 3.0 |
| Glycerin | Glycerin | 10.0 |
| Betaine | Betaine | 5.0 |
| PEG 1500 | PEG 1500 | 1.5 |
| Allatoin | Allatoin | 0.3 |
| DL-Panthenol | DL-Panthenol | 0.5 |
| EDTA-2Na | EDTA-2Na | 0.02 |
| Benzopyrene-9 | Benzopyrene-9 | 0.05 |
| Hydroxymethylcellulose | Hydroxymethylcellulose | 0.1 |
| Sodium hyaluronate | Sodium hyaluronate | 10.0 |
| Carboxyvinyl polymer | Carboxyvinyl polymer | 0.2 |
| Trimethanolamine | Trimethanolamine | 0.2 |
| Octyldodecanol | Octyldodecanol | 0.3 |
| Octyldodeceth-16 | Octyldodeceth-16 | 0.4 |
| Ethanol | Ethanol | 8.0 |
| Preservative, | Preservative, | Small amount |
| fragrance, pigment | fragrance, pigment | |
| Distilled water | Distilled water | Residual amount |

| | |
|---|---|
| Sum | 100 |

(4) Evaluation of Cytotoxicity

The skin-lifting cosmetic compositions for anti-aging prepared in Formulation Examples 1 to 3, according to the present disclosure, were used to measure cytotoxicity in HaCaT cells and HDF-N cells (see Table 4 below). The HaCaT cells were dispensed in a 96-well plate at a concentration of 5×10⁴ cells per well, and the HDF-N cells were dispensed in a 96-well plate at a concentration of 6×10³ cells per well, followed by being cultured for 24 hours under cell culture conditions. After removing the media, washing the cultured cells with DPBS, and maintaining a starvation state for 12 hours, a test solution and new media (free of a media supplement) were added, and incubation was performed for 24 hours. In addition, to measure survival rates of the cells, after removing the test solution and the media, and washing the cultured cells with PBS, a WST-1 reaction solution was diluted to a scale of 1/10 in a supplement-free medium, and then the diluted solution at a volume of 100 μl per well was treated to cause reactions for 1 hour. Next, the absorbance was measured at a wavelength of 450 nm. As a result, when the concentrations were up to 100 ppm in the HaCaT cells and the HDF-N cells, it was confirmed that Formulation Example 1 exhibited survival rates of 99.4% and 98.6%, Formulation Example 2 exhibited survival rates of 99.5% and 98.4%, and Formulation Example 3 exhibited survival rates of 99.6% and 98.2%, as shown in Table 4.

TABLE 4

| | Control | Experimental group (Cytotoxicity | | | | | |
|---|---|---|---|---|---|---|---|
| Specimen | group | to HaCaT cells) | | | | | |
| Concentration | 0 | 1 | 3 | 5 | 10 | 50 | 100 |
| (ppm) | | | | | | | |
| Formulation | 100 | 100.0 | 99.4 | 99.2 | 99.3 | 99.4 | 99.4 |
| Example 1 | | | | | | | |
| Formulation | 100 | 100.1 | 99.8 | 99.6 | 99.2 | 99.8 | 99.5 |
| Example 2 | | | | | | | |
| Formulation | 100 | 99.9 | 99.9 | 99.7 | 99.8 | 99.6 | 99.6 |
| Example 3 | | | | | | | |
| | Control | Experimental group (Cytotoxicity | | | | | |
| Specimen | group | to HDF-N cells) | | | | | |
| Concentration | 0 | 1 | 3 | 5 | 10 | 50 | 100 |
| Formulation | 100 | 100.1 | 99.8 | 98.6 | 98.2 | 98.0 | 98.6 |
| Example 1 | | | | | | | |
| Formulation | 100 | 100.0 | 99.9 | 99.4 | 99.3 | 99.2 | 98.4 |
| Example 2 | | | | | | | |
| Formulation | 200 | 99.8 | 98.6 | 98.9 | 99.2 | 99.4 | 89.2 |
| Example 3 | | | | | | | |

As described above, it was confirmed that the toner, the cream, and the essence among the cosmetics, that is, the skin-lifting cosmetic composition for anti-aging according to the present disclosure, containing the core-shell particles including the gold (Au) nanoparticle layer as the active ingredient did not exhibit cytotoxicity, and thus had excellent stability to the skin.

### <Example 2> Implementation of Photothermal Skin Care Method Using Cosmetic Composition of Example 1

A clinical trial was performed on patients with advanced aging (Class 3) on the stage-based classification of photoaging skin damage. The classification of skin damage was based on the Glogau Photodamage Classification Scale (Table 5). Class 3 and higher, mainly middle-aged people of age not fewer than a range of 50 years to 65 years, correspond to faces on which wrinkles are visible even when at rest, and a foundation is required to be heavily applied due to obvious pigmentation, noticeable telangiectasia, keratosis, and the like.

TABLE 5

| | | Typical | | |
|---|---|---|---|---|
| Group | Classification | Age | Description | Skin Characteristics |
| 1 | Mild | 28 to 35 | No wrinkles | Early photoaging: mild |
| | | | | pigment changes, no |
| | | | | keratosis, minimal |
| | | | | wrinkles, minimal or no |
| | | | | makeup |
| 2 | Moderate | 35 to 50 | Wrinkles | Early to moderate |
| | | | in motion | photoaging: early brown |
| | | | | spots visible, Keratosis |
| | | | | palpable but not visible, |
| | | | | smile lines begin to |
| | | | | appear, wears some |
| | | | | foundation |
| 3 | Advanced | 50 to 65 | Wrinkles | Advanced photoaging: |
| | | | at rest | obvious discolorations, |
| | | | | telangiectasia, visible |
| | | | | keratosis, wears heavier |
| | | | | foundation always |
| 4 | Severe | 60 to 75 | Only | Severe photoaging: yellow- |
| | | | wrinkles | gray skin color, prior |
| | | | | skin malignancies, wrinkles |
| | | | | everywhere, no normal |
| | | | | skin, caking and creasing |
| | | | | due to heavy makeup |

First, the facial skin of a subject was peeled through superficial chemical peeling using a 2% aqueous solution of trichloroacetic acid (TCA) so that an active ingredient of a cosmetic composition was well delivered to the follicles, sebaceous glands, pilosebaceous ducts, and infundibula of the skin.

Subsequently, the cosmetic composition was applied on the facial skin of the subject at a volume in a range of about 6 ml to 7 ml prepared in Example 1.

Next, a step of primarily facilitating infiltration of core-shell particles contained in the cosmetic composition into the follicles, sebaceous glands, pilosebaceous ducts, and infundibula of the skin by applying 1-MHz ultrasound to the facial skin of the subject, having undergone the application step, for 5 minutes to 10 minutes was performed. Thereafter, a step of secondarily facilitating in which a face-shaped vinyl film was formed and then attached to the face in a mask form was performed to further facilitate the absorption of the core-shell particles into the skin.

Then, a step of removing residues of the cosmetic composition with a Q-switched Nd:YAG laser beam was performed on the skin of the subject, having undergone the above facilitation steps, to remove the residues of the core-shell particles.

Thereafter, the cosmetic composition-removed facial skin was irradiated with an intense pulsed light (IPL) laser using a combined machine of long-pulsed 755-nm alexandrite laser and 1064-nm ND:YAG laser to heat the core-shell particles infiltrated into the follicles, sebaceous glands, pilosebaceous ducts, and infundibula of the skin, thereby inducing a skin-lifting effect.

Irradiation conditions are as follows.
A wavelength of 755 nm
A spot size of 10 mm
A pulse width of 30 ms
An energy fluence in a range of 15 J/cm² to 20 J/cm²
A frequency of 1.5 Hz (300 shots to 450 shots)

The experiment results were obtained by evaluating the degree of elasticity improvement with the Dermal Torque Meter, a skin measurement device.

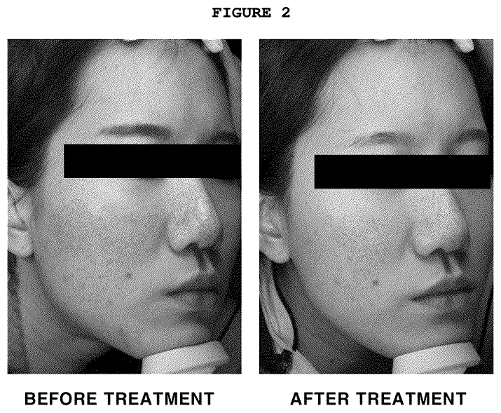

It is seen that FIG. 2 shows the conditions of the test subject before conducting the experiment (left) and after performing the treatment two times using the method of the present disclosure (right). It is confirmed that even with the bare eye, the number of pores shown as spots is significantly reduced after the treatment.

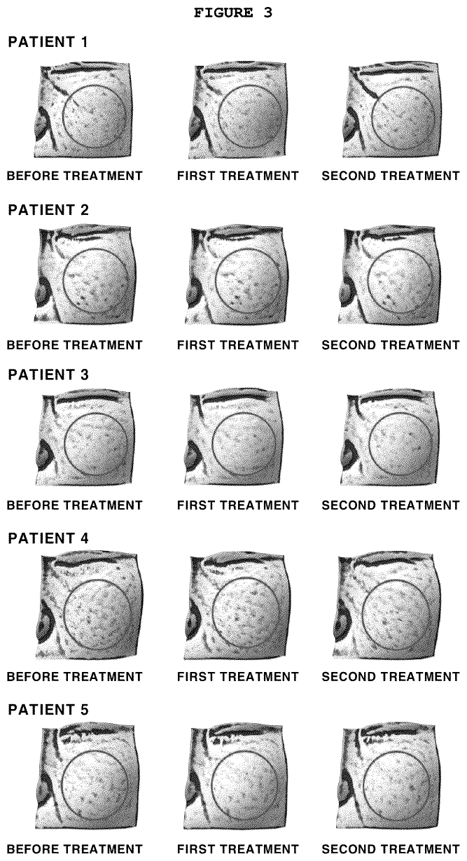

FIG. 3 shows the skin conditions of each patient before performing the treatment and after performing the treatment one time and two times, in which the deeper the wrinkles, the greener the skin, and the lighter the wrinkles, the yellower the skin. It is confirmed that the depth of wrinkles on the epidermis of the patient is reduced, thereby increasing elasticity. As a result of analyzing the degree of improvement in deep skin elasticity, it was seen that the elasticity after the treatment was increased to a meaningful level, compared to that before the treatment. The Ur/Ue value, an indicator of skin elasticity, was 5.31% after the first treatment and 8.80% after the second treatment, thereby exhibiting an elasticity improvement effect compared to that before the treatment.

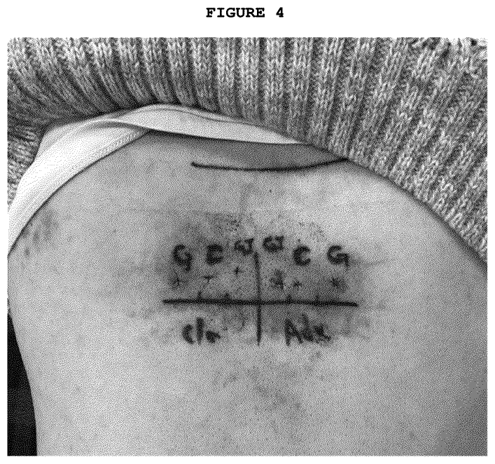

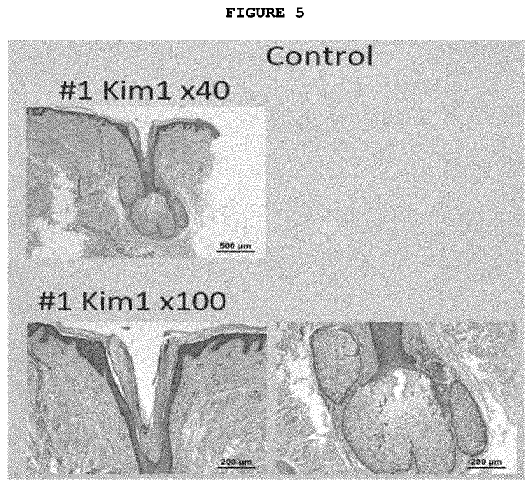

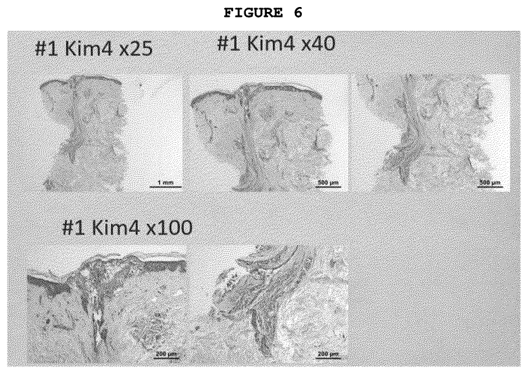

FIG. 4 shows an image of a tissue test on the back of a biopsy subject where gold is delivered into pores and shrinks the pores by converting a laser beam into heat. FIG. 5 shows a hematoxylin-eosin (H&E) staining image of the pores before performing the treatment, and FIG. 6 shows an H&E staining image of the pores after performing the treatment. It is seen that the pores after the treatment are in a significantly improved condition compared to the pores damaged by the heat before the treatment.

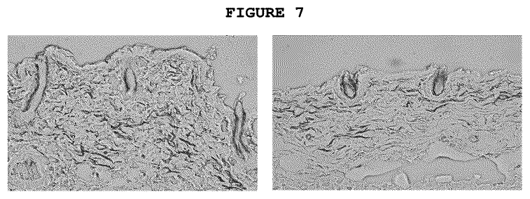

To confirm the effect of the occlusive dressing technique (ODT) in the present disclosure, tissue changes were observed when irradiating a hairless mouse only with ultrasound (left) and a laser beam in a combination of laser application and ODT treatment (right). The results thereof are shown in FIG. 7. In the tissue image, round areas represent the pores and indicate the degree of pore shrinkage. Looking into the left image, when irradiated with only the ultrasound, the pores failed to be shrunk completely. On the other hand, it was confirmed that when irradiated with the laser beam in a combination of the ultrasound application and the ODT treatment, the pores were shrunk completely.

### <Example 3> Transmission Electron Microscopy Images

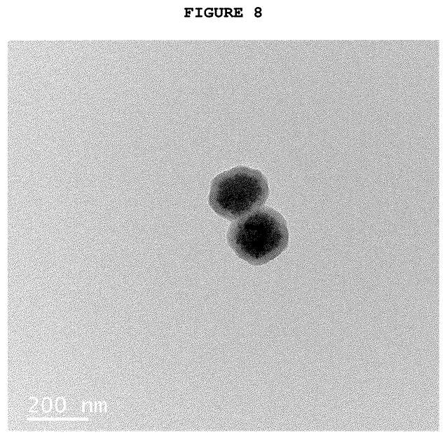

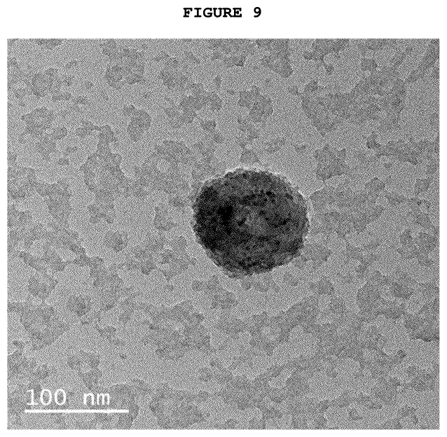

FIGS. 8 and 9 show transmission electron microscopy (TEM) images of particles coated with only silica and particles coated with gold on top of a silica layer, respectively. Through the results, it was confirmed that the particles were prepared at a size of about 150 nm. Thus, the present disclosure has a technical purpose contrary to delivering an active ingredient into the skin. In addition, it was confirmed that the cosmetic composition, according to the present disclosure, was prepared to prevent a magnetic substance in a second shell layer from being absorbed into the skin.

Although the embodiment of the present disclosure has been described with reference to the accompanying drawings, those skilled in the art will appreciate that various modifications, additions and substitutions are possible, without departing from the scope and spirit of the disclosure as disclosed in the accompanying claims. It is thus well known to those skilled in that art that the present disclosure is not limited to the embodiment disclosed in the detailed description.

1. A skin-lifting cosmetic composition for anti-aging, the composition comprising core-shell particles as an active ingredient,
wherein the core-shell particle comprises:
a magnetic core;
a first shell layer surrounding the magnetic core and comprising a dielectric substance;
a second shell layer surrounding the first shell layer and comprising one or more metals selected from the group consisting of gold (Au), silver (Ag), and platinum (Pt); and
a third shell layer surrounding the second shell layer and comprising a hydrophilic polymer.

2. The cosmetic composition of claim 1, wherein the dielectric substance comprised in the first shell layer is silica, the metal comprised in the second shell layer is gold (Au), and the hydrophilic polymer comprised in the third shell layer is polyethylene glycol (PEG).

3. The cosmetic composition of claim 1, wherein the core-shell particles are comprised in an amount of 0.1 mg/mL to 10 mg/mL based on the total volume of the cosmetic composition.

4. A photothermal skin care method for skin lifting, the method comprising:
(a) peeling facial skin;
(b) applying the cosmetic composition of claim 1 on the surface of the facial skin;
(c) primarily facilitating infiltration of core-shell particles comprised in the cosmetic composition into follicles, sebaceous glands, pilosebaceous ducts, and infundibula of the skin by applying ultrasound to the facial skin coated with the cosmetic composition;
(d) secondarily facilitating infiltration of the core-shell particles comprised in the cosmetic composition into the follicles, sebaceous glands, pilosebaceous ducts, and infundibula of the skin by attaching a polymer film mask to the facial skin coated with the cosmetic composition;
(e) removing residues of the cosmetic composition from the surface of the facial skin with a laser beam; and
(f) heating the core-shell particles infiltrated into the follicles, sebaceous glands, pilosebaceous ducts, and infundibula of the skin by irradiating the cosmetic composition-removed facial skin with light.

5. The method of claim 4, wherein in the (a), superficial chemical peeling using trichloroacetic acid (TCA) is performed to peel the facial skin.

6. The method of claim 4, wherein in the (e), the facial skin is irradiated with a Q-switched Nd:YAG laser beam to remove the residues of the cosmetic composition from the surface of the facial skin.
